(19) United States
(12) Patent Application Publication
(10) Pub. No.: US 2023/0352155 A1
(43) Pub. Date: Nov. 2, 2023

(54) METHOD AND SYSTEM FOR FORECASTING DEMAND FOR NURSING SERVICES

(71) Applicants: Dagnachew Birru; Timothy Elwell; Sofia P. Moschou; Srinivas Kulkarni

(72) Inventors: Dagnachew Birru; Timothy Elwell; Sofia P. Moschou; Srinivas Kulkarni

(73) Assignee: Quantiphi, Inc, Marlborough, MA (US)

(21) Appl. No.: 18/219,651

(22) Filed: Jul. 8, 2023

(51) Int. Cl. G16H 40/20; G06Q 10/0631

(52) U.S. Cl. CPC G16H 40/20; G06Q 10/06315; G06Q 10/06312

(57) ABSTRACT

A method and system for forecasting demand for nursing services in a hospital herein. The method and system comprises accessing external data and historical data of a hospital. The method and system further comprises combining the external data and historical data of a hospital to form a structured data aggregation. Further, the structured data aggregation is processed. The method and system further comprises forecasting for a time interval, the demand for nursing services. In addition, work drivers are an accurate workload indicator for nurse workload planning and takes into consideration patient-dependent diversified needs. Furthermore, the system comprises a productivity index that accounts for nurses down-time and administrative tasks that need to be subtracted from the time spent on clinical tasks. This is used to schedule accurately the nurse rota based on realistic hospital needs and uses historic data to predict the upcoming demand over different ranges of time-horizons.

### TECHNICAL FIELD OF THE INVENTION

The present disclosure is related to method and system for forecasting demand for nursing services in a hospital.

### BACKGROUND OF THE INVENTION

In a hospital setting, nursing services play a crucial role in providing quality patient care. Accurate demand forecasting for nursing services is essential for effective resource allocation, proper staffing levels, and maintaining optimal patient outcomes. This disclosure explores the key steps and factors involved in nursing services demand forecasting within a hospital setting.

The first step in nursing services demand forecasting is analyzing historical data. This includes reviewing data related to patient admissions, length of stay, patient acuity levels, weather events, and nursing workload. By studying past trends, hospitals can identify patterns and variations that help in predicting future demand accurately.

Estimating patient volume is fundamental in determining nursing service demand. Hospitals should consider historical trends, population demographics, and any upcoming events or seasonal variations that may impact patient admissions. By factoring in these elements, hospitals can anticipate fluctuations in patient numbers and adjust nursing resources accordingly.

Assessing patient acuity levels is vital for forecasting nursing service demand accurately. Patient acuity refers to the complexity and intensity of care required by patients. Higher acuity patients typically need more nursing care and resources. Historical data, clinical expertise, and input from healthcare professionals are invaluable in determining the expected distribution of patient acuity levels, enabling hospitals to allocate appropriate nursing resources.

Analyzing historical data helps in estimating the average length of stay for different patient categories. Patient length of stay impacts nursing service demand since longer stays require sustained nursing care, while shorter stays may result in higher patient turnover. By understanding length of stay patterns, hospitals can better plan nursing staffing schedules and allocate resources accordingly.

Determining appropriate staffing ratios is critical for maintaining quality patient care. Hospitals must consider nurse-to-patient ratios, nurse-to-specialty ratios, and specific unit or department requirements. By evaluating workload associated with different patient acuity levels, hospitals can optimize staffing needs and ensure nurses are not overburdened, leading to better patient outcomes.

Forecasting nursing service demand requires considering external factors that may impact patient admissions. Changes in healthcare policies, reimbursement models, population growth or decline, weather and disease outbreaks can significantly influence demand. Hospitals should stay informed about local and national healthcare trends to adjust their forecasting models accordingly.

Anticipating nursing service demand also involves assessing planned or ongoing changes in healthcare technology, workflows, or processes. Implementing electronic health records or new care delivery models may impact nursing workload. By considering these changes, hospitals can accurately forecast demand and align staffing levels with evolving healthcare practices.

Engaging nursing managers, nursing leadership, frontline nurses, and other healthcare professionals is vital in the demand forecasting process. Their valuable insights regarding unit-specific demands, upcoming projects, or changes in patient care requirements can significantly improve the accuracy of forecasting models.

Utilizing forecasting models and techniques, such as time series analysis, regression analysis, or predictive modeling, can enhance nursing service demand forecasts. These models help identify patterns, trends, and correlations within the collected data, resulting in more accurate predictions.

Continuous monitoring and evaluation of nursing service demand against actual data is crucial. This allows hospitals to assess the accuracy of forecasts and make necessary adjustments to improve future predictions. By incorporating feedback and new data, hospitals can refine their forecasting strategies and optimize resource allocation.

Nursing services demand forecasting in a hospital setting is a complex process that requires thorough analysis of historical data, patient volume forecasting, patient acuity assessment, length of stay prediction, staffing ratios and workload analysis, consideration of external influences, technology and process changes, collaboration with healthcare professionals, and the use of forecasting models. Accurate forecasting enables hospitals to efficiently allocate. However, the existing solutions are not able to provide accurate demand forecasting due to complex scenarios.

It is within this context that the present embodiments arise.

### SUMMARY

The following embodiments present a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Some example embodiments disclosed herein provide a method for forecasting demand for nursing services in a hospital, the method comprising accessing historical data of the hospital. The method may further include accessing nursing notes and accessing external data. The method may include combining the external data and the historical data of the hospital to form a structured data aggregation. The method may include processing the structured data aggregation. The method may also include forecasting for a time interval, the demand for nursing services based on the processed structured data aggregation.

According to some example embodiments, the historical data of the hospital comprises of at least patient-level work drivers, departmental data, ICD-10 data, and day of a week the score is calculated based on syntactic similarity.

According to some example embodiments, the external data comprises of at least historical pandemic data, seasonal communicable disease data and weather data.

According to some example embodiments, processing the structured data aggregation comprises cleaning the structured data aggregation. The method may include normalizing the structured data aggregation. The method may include performing exploratory data analysis of the structured data aggregation. The method may also include executing feature engineering of the structured data aggregation.

According to some example embodiments, the time interval comprises 4 hours, 8 hours, 12 hours and 24 hours.

According to some example embodiments, the forecasting is performed by applying XGBoost algorithm on the processed structured data aggregation to produce a plurality of candidate forecasts.

According to some example embodiments, the method further comprised obtaining an optimum forecast by hyper-parameter tuning of the plurality of candidate forecasts.

According to some example embodiments, the forecasting is online and real-time.

According to some example embodiments, the forecasting further comprises clustering based on historical data.

Some example embodiments disclosed herein provide a computer system for forecasting demand for nursing services in a hospital, the computer system comprises one or more computer processors, one or more computer readable memories, one or more computer readable storage devices, and program instructions stored on the one or more computer readable storage devices for execution by the one or more computer processors via the one or more computer readable memories, the program instructions comprising accessing historical data of the hospital. The one or more processors are further configured to accessing external data. The one or more processors are configured to combining the external data and the historical data of the hospital to form a structured data aggregation. The one or more processors are configured to processing the structured data aggregation. The one or more processors are further configured to forecasting for a time interval, the demand for nursing services based on the processed structured data aggregation.

Some example embodiments disclosed herein provide a non-transitory computer readable medium having stored thereon computer executable instruction which when executed by one or more processors, cause the one or more processors to carry out operations for forecasting demand for nursing services in a hospital. The operations comprising accessing historical data of the hospital. The operations further comprising accessing external data. The operations comprising combining the external data and the historical data of the hospital to form a structured data aggregation. The operations comprising processing the structured data aggregation. The operations further comprising forecasting for a time interval, the demand for nursing services based on the processed structured data aggregation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The above and still further example embodiments of the present disclosure will become apparent upon consideration of the following detailed description of embodiments thereof, especially when taken in conjunction with the accompanying drawings, and wherein:

FIG. 1 illustrates a block diagram for forecasting demand for nursing services in a hospital, in accordance with an example embodiment;

FIG. 2 illustrates a block diagram of an electronic circuitry for forecasting demand for nursing services, in accordance with an example embodiment;

FIG. 3 illustrates a block diagram representing data handling with regards to forecasting demand for nursing services, in accordance with an example embodiment;

FIG. 4 illustrates a block diagram that shows various input data used in forecasting demand, in accordance with an example embodiment;

FIG. 5 illustrates a block diagram that shows different components in data processing module, in accordance with an example embodiment;

FIG. 6 illustrates a block diagram that shows various forecasting models, in accordance with an example embodiment;

FIG. 7 illustrates a block diagram that shows various components in approaches to demand forecasting, in accordance with an example embodiment;

FIG. 8 shows a flow diagram of a method for forecasting the demand for nursing services, in accordance with an example embodiment;

FIG. 9 shows a flow diagram of a method for demand input data processing, in accordance with an example embodiment;

FIG. 10 shows a flow diagram of a method for execution of Machine Learning model, in accordance with an example embodiment;

FIG. 11 shows a block diagram for process flow of nursing services demand prediction, in accordance with an example embodiment;

The figures illustrate embodiments of the invention for purposes of illustration only. One skilled in the art will readily recognize from the following description that alternative embodiments of the structures and methods illustrated herein may be employed without departing from the principles of the invention described herein.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, systems, apparatuses, and methods are shown in block diagram form only in order to avoid obscuring the present invention.

Reference in this specification to “one embodiment” or “an embodiment” or “example embodiment” means that a particular feature, structure, or characteristic described in connection with the embodiment is included in at least one embodiment of the present invention. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout.

The terms “comprise”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present invention. Further, it is to be understood that the phraseology and terminology employed herein are for the purpose of the description and should not be regarded as limiting. Any heading utilized within this description is for convenience only and has no legal or limiting effect.

### Definitions

The term “module” used herein may refer to a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof.

The term “machine learning model” may be used to refer to a computational or statistical or mathematical model that is trained on classical ML modelling techniques with or without classical image processing. The “machine learning model” is trained over a set of data and using an algorithm that it may use to learn from the dataset.

The term “artificial intelligence” may be used to refer to a model built using simple or complex Neural Networks using deep learning techniques and computer vision algorithms. Artificial intelligence model learns from the data and applies that learning to achieve specific pre-defined objectives.

### End of Definitions

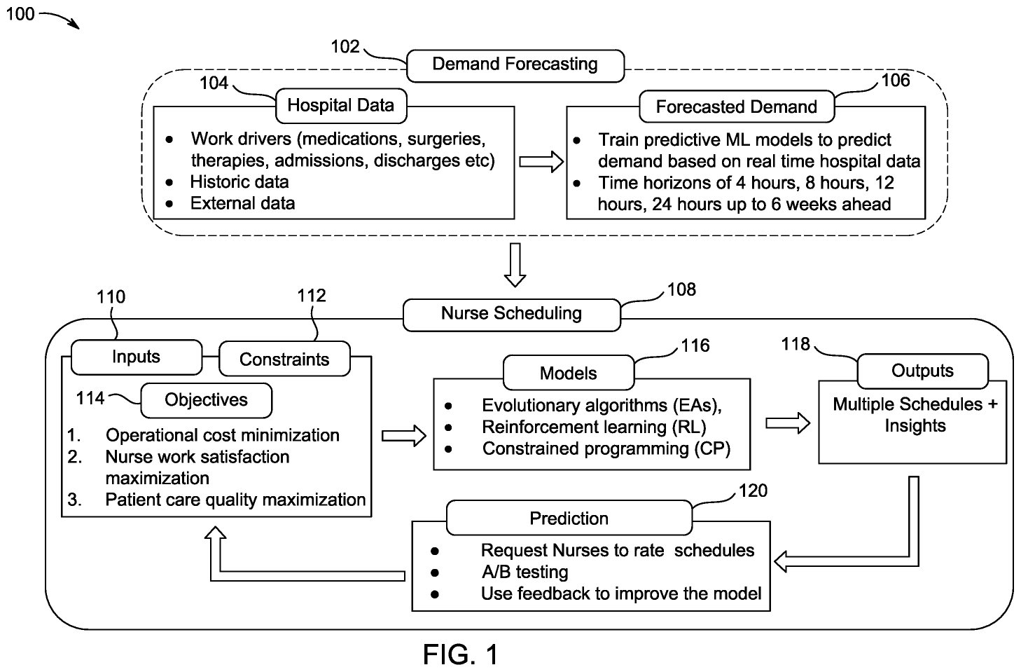

Embodiments of the present disclosure may provide a method, a system, and a computer program product for forecasting demand for nursing services in a hospital The method, the system, and the computer program product for forecasting demand for nursing services in a hospital are described with reference to FIG. 1 to FIG. 10 as detailed below.

Nursing services demand forecasting in a hospital setting is a critical aspect of healthcare management. Accurately predicting the future demand for nursing services is essential for hospitals to allocate resources efficiently, plan staffing schedules effectively, and ensure the delivery of high-quality patient care. The key steps and factors involved in nursing services demand forecasting within a hospital setting are mentioned below.

To begin with, historical data analysis is a fundamental step in nursing services demand forecasting. Hospitals need to analyze data related to patient admissions, length of stay, patient acuity levels, and nursing workload. By studying past trends and patterns, hospitals can gain insights into the variations and fluctuations in nursing service demand, which helps in predicting future demand more accurately.

Patient volume forecasting is a crucial aspect of nursing services demand forecasting. Hospitals must consider historical trends, population demographics, and any upcoming events or seasonal variations that may impact patient admissions. By taking these factors into account, hospitals can estimate fluctuations in patient numbers and adjust nursing resources accordingly.

Predicting the length of stay for different patient categories is also crucial in nursing services demand forecasting. By analyzing historical data, hospitals can estimate the average length of stay. This information is essential in determining nursing staffing needs, as longer stays require sustained nursing care, while shorter stays may result in higher patient turnover. Accurate length of stay prediction allows hospitals to plan staffing schedules and allocate resources effectively.

External influences are important considerations in nursing services demand forecasting. Changes in healthcare policies, reimbursement models, population growth or decline, and disease outbreaks can significantly impact demand for nursing services. Hospitals need to stay informed about local and national healthcare trends to adjust their forecasting models accordingly and accurately predict nursing service demand.

Furthermore, forecasting nursing service demand requires considering technology and process changes. Implementation of electronic health records or new care delivery models can impact nursing workload. Hospitals need to assess planned or ongoing changes in healthcare technology, workflows, or processes and incorporate these factors into their demand forecasting models.

Collaboration and expert input are invaluable in the nursing services demand forecasting process. Engaging nursing managers, frontline nurses, and other healthcare professionals allows for the incorporation of their insights regarding unit-specific demands, upcoming projects, or changes in patient care requirements. This collaboration improves the accuracy of forecasting models and ensures that nursing service demand is projected more effectively.

Utilizing forecasting models and techniques is essential in nursing services demand forecasting. Time series analysis, regression analysis, and predictive modeling can help identify patterns, trends, and correlations within the collected data. These models enhance the accuracy of predictions and enable hospitals to make informed decisions regarding resource allocation and staffing.

Furthermore, regular monitoring and evaluation are vital in nursing services demand forecasting. Hospitals need to continuously monitor the accuracy of their forecasts against actual nursing service demand. By comparing forecasts with real-time data, hospitals can assess the effectiveness of their forecasting models and make necessary adjustments to improve future predictions.

Therefore, nursing services demand forecasting in a hospital setting is a complex process that involves analyzing historical data, forecasting patient volume and length of stay, assessing patient acuity, analyzing staffing ratios and workload, considering external influences, incorporating technology and process changes, collaborating with healthcare professionals, and utilizing forecasting models. Accurate demand forecasting enables hospitals to allocate resources efficiently, plan staffing schedules effectively, and ensure the delivery of high-quality patient care.

Accordingly, the present disclosure provides a method, system, or computer program product for forecasting demand for nursing services in a hospital.

FIG. 1 illustrates a block diagram for forecasting demand for nursing services in a hospital, in accordance with an example embodiment.

In an embodiment, a hospital has a system 100, which is further divided into a demand forecasting module 102 and a nurse scheduling module 108.

Further, the demand forecasting module 102 utilizes hospital data 104 and produces the forecasted demand 106.

In an example embodiment, hospital data 104 plays a crucial role in nursing services demand forecasting. Further, the hospital data may comprise of work drivers, historic data and external data. Also, the work drivers may include medications, nursing notes, surgeries, therapies, admissions and discharges. To accurately predict future demand for nursing services, hospitals rely on various types of data that provide insights into patient admissions, length of stay, patient acuity levels, nursing workload, and other relevant factors.

Firstly, patient admission data is essential for understanding the overall volume of patients entering the hospital. This data includes information about the number of patients admitted to the hospital within a specific period. By analyzing historical patient admission data, hospitals can identify trends, patterns, and variations in patient volumes, enabling them to anticipate future demand for nursing services.

Length of stay data provides insights into the duration of patients' hospital stays. Hospitals track the length of stay for each patient, which helps in estimating the average length of stay for different patient categories or medical conditions. This data is crucial for determining the nursing resources required to provide care for patients during their hospitalization.

In an example embodiment, patient acuity data is another vital component of nursing services demand forecasting. Acuity refers to the complexity and intensity of care required by patients. Hospitals collect data on patient acuity levels, which can be determined based on various factors such as medical conditions, treatments, and interventions. Understanding the distribution of patient acuity levels allows hospitals to allocate nursing resources according to the level of care required by patients.

In an example embodiment, ICD-10 coding and co-morbidities may be employed to predict acuity. Further, the relationship of acuity to orders may be used to determine and estimate work estimates per patient driven by historical patterns by unit and day of the week.

In an example embodiment, nursing workload data provides insights into the tasks and activities performed by nursing staff. This data helps in assessing the amount of time and effort required by nurses to deliver care to patients. By analyzing nursing workload data, hospitals can determine the appropriate staffing levels and nurse-to-patient ratios to ensure adequate coverage and high-quality care.

In addition to these specific patient-related data, hospitals also consider external factors that may influence nursing services demand. This includes data related to healthcare policies, reimbursement models, population demographics, and disease outbreaks. Monitoring and analyzing these external factors help hospitals make informed projections about future demand for nursing services.

Technology and process data are also important in nursing services demand forecasting. Hospitals collect data on the implementation of healthcare technologies, such as electronic health records or automated systems, which can impact nursing workflows and workload. Understanding the impact of technological advancements and process changes allows hospitals to adjust their forecasting models to account for these factors.

Collaborative data involving the input of nursing managers, frontline nurses, and other healthcare professionals is invaluable in nursing services demand forecasting. These individuals provide valuable insights into unit-specific demands, upcoming projects, or changes in patient care requirements. By incorporating their expertise and experience, hospitals can enhance the accuracy of their demand forecasting models.

To analyze and make sense of this vast amount of data, hospitals often employ advanced analytics techniques and forecasting models. These models help identify patterns, trends, and correlations within the collected data, improving the accuracy of demand forecasts.

Therefore, nursing services demand forecasting in hospitals relies on a diverse range of data. Patient admission data, length of stay data, patient acuity data, nursing workload data, external factors data, technology and process data, and collaborative data all play crucial roles in accurately predicting the future demand for nursing services. By analyzing and incorporating these types of data into forecasting models, hospitals can effectively allocate resources, plan staffing schedules, and ensure high-quality patient care.

In another example embodiment, the output forecasted demand 106 may be achieved by training a predictive machine learning model based on the hospital data 104. Forecasting may be achieved for time slices of 4,8, 12 and 24 hours up to 6 weeks ahead.

In an embodiment, nurse scheduling 108 has multiple modules such as inputs 110, constraints 110, objectives 114, models 116, outputs 118 and nurse survey feedback 120

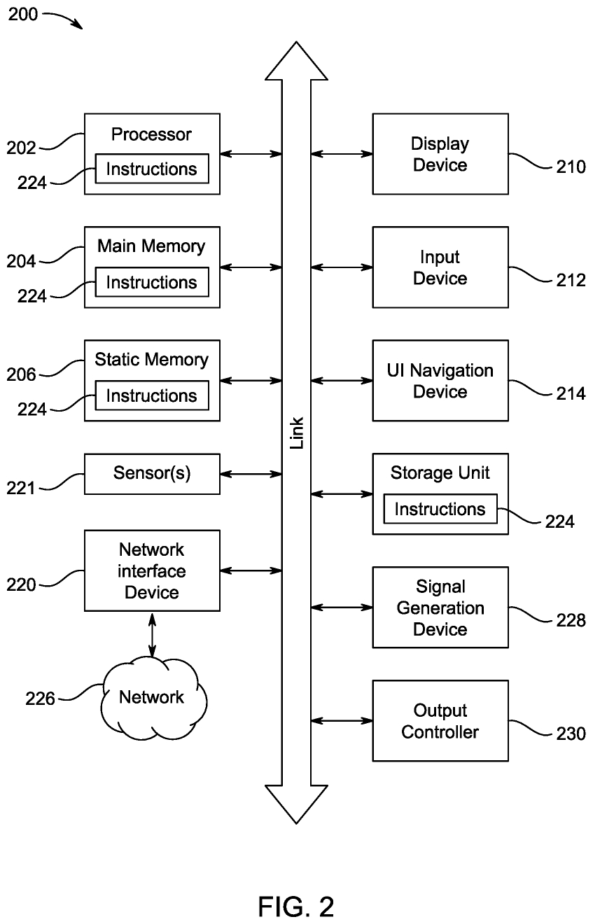

FIG. 2 illustrates a block diagram of an electronic circuitry for identifying optimal utterances for virtual agent training. The machine of FIG. 2 is shown as a standalone device, which is suitable for implementation of the concepts above. For the server aspects described above a plurality of such machines operating in a data center, part of a cloud architecture, and so forth can be used. In server aspects, not all of the illustrated functions and devices are utilized. For example, while a system, device, etc. that a user uses to interact with a server and/or the cloud architectures may have a screen, a touch screen input, etc., servers often do not have screens, touch screens, cameras and so forth and typically interact with users through connected systems that have appropriate input and output aspects. Therefore, the architecture below should be taken as encompassing multiple types of devices and machines and various aspects may or may not exist in any particular device or machine depending on its form factor and purpose (for example, servers rarely have cameras, while wearables rarely comprise magnetic disks). However, the example explanation of FIG. 2 is suitable to allow those of skill in the art to determine how to implement the embodiments previously described with an appropriate combination of hardware and software, with appropriate modification to the illustrated embodiment to the particular device, machine, etc. used.

While only a single machine is illustrated, the term “machine” shall also be taken to include any collection of machines that individually or jointly execute a set (or multiple sets) of instructions to perform any one or more of the methodologies discussed herein.

The example of the machine 200 includes at least one processor 202 (e.g., a central processing unit (CPU), a graphics processing unit (GPU), advanced processing unit (APU), or combinations thereof), one or more memories such as a main memory 204, a static memory 206, or other types of memory, which communicate with each other via link 208. Link 208 may be a bus or other type of connection channel. The machine 200 may include further optional aspects such as a graphics display unit 210 comprising any type of display. The machine 200 may also include other optional aspects such as an alphanumeric input device 212 (e.g., a keyboard, touch screen, and so forth), a user interface (UI) navigation device 214 (e.g., a mouse, trackball, touch device, and so forth), a storage unit 216 (e.g., disk drive or other storage device(s)), a signal generation device 218 (e.g., a speaker), sensor(s) 221 (e.g., global positioning sensor, accelerometer(s), microphone(s), camera(s), and so forth), output controller 228 (e.g., wired or wireless connection to connect and/or communicate with one or more other devices such as a universal serial bus (USB), near field communication (NFC), infrared (IR), serial/parallel bus, etc.), and a network interface device 220 (e.g., wired and/or wireless) to connect to and/or communicate over one or more networks 226.

Executable Instructions and Machine-Storage Medium

The various memories (i.e., 204, 206, and/or memory of the processor(s) 202) and/or storage unit 216 may store one or more sets of instructions and data structures (e.g., software) 224 embodying or utilized by any one or more of the methodologies or functions described herein. These instructions, when executed by processor(s) 202 cause various operations to implement the disclosed embodiments.

Example Machine Architecture and Machine-Readable Medium

FIG. 2 illustrates a representative machine architecture suitable for implementing the systems and so forth or for executing the methods disclosed herein. The machine of FIG. 2 is shown as a standalone device, which is suitable for implementation of the concepts above. For the server aspects described above a plurality of such machines operating in a data center, part of a cloud architecture, and so forth can be used. In server aspects, not all of the illustrated functions and devices are utilized. For example, while a system, device, etc. that a user uses to interact with a server and/or the cloud architectures may have a screen, a touch screen input, etc., servers often do not have screens, touch screens, cameras and so forth and typically interact with users through connected systems that have appropriate input and output aspects. Therefore, the architecture below should be taken as encompassing multiple types of devices and machines and various aspects may or may not exist in any particular device or machine depending on its form factor and purpose (for example, servers rarely have cameras, while wearables rarely comprise magnetic disks). However, the example explanation of FIG. 2 is suitable to allow those of skill in the art to determine how to implement the embodiments previously described with an appropriate combination of hardware and software, with appropriate modification to the illustrated embodiment to the particular device, machine, etc. used.

While only a single machine is illustrated, the term “machine” shall also be taken to include any collection of machines that individually or jointly execute a set (or multiple sets) of instructions to perform any one or more of the methodologies discussed herein.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include storage devices such as solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage media, computer-storage media, and device-storage media specifically and unequivocally excludes carrier waves, modulated data signals, and other such transitory media, at least some of which are covered under the term “signal medium” discussed below.

Signal Medium

The term “signal medium” shall be taken to include any form of modulated data signal, carrier wave, and so forth. The term “modulated data signal” means a signal that has one or more of its characteristics set or changed in such a matter as to encode information in the signal.

Computer Readable Medium

The terms “machine-readable medium,” “computer-readable medium” and “device-readable medium” mean the same thing and may be used interchangeably in this disclosure. The terms are defined to include both machine-storage media and signal media. Thus, the terms include both storage devices/media and carrier waves/modulated data signals.

As used herein, the term “network” may refer to a long-term cellular network (such as GSM (Global System for Mobile Communication) network, LTE (Long-Term Evolution) network or a CDMA (Code Division Multiple Access) network) or a short-term network (such as Bluetooth network, Wi-Fi network, NFC (near-field communication) network, LoRaWAN, ZIGBEE or Wired networks (like LAN, el all) etc.).

As used herein, the term “computing device” may refer to a mobile phone, a personal digital assistance (PDA), a tablet, a laptop, a computer, VR Headset, Smart Glasses, projector, or any such capable device.

As used herein, the term ‘electronic circuitry’ may refer to (a) hardware-only circuit implementations (for example, implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

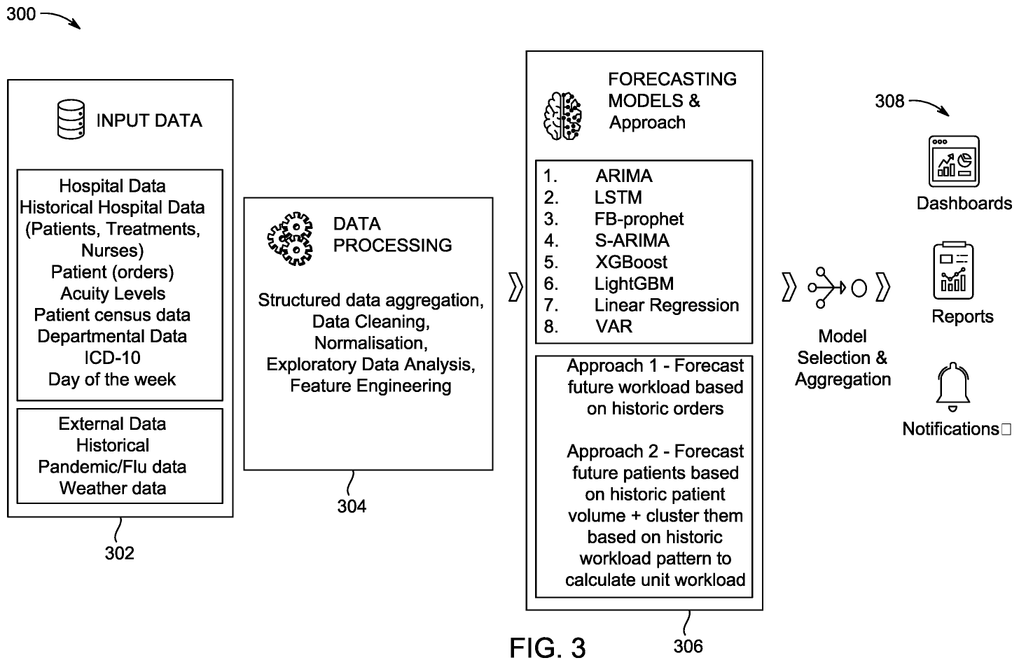

FIG. 3 illustrates a block diagram representing data handling with regards to forecasting demand for nursing services, in accordance with an example embodiment. The principal components of data handling system 300 are input data 302, data processing module 304, forecasting models 306 and output 308.

In an example embodiment, the output 308 is in the form of dashboards, reports, and notifications. Furthermore, the output 308 estimation includes a productivity index that accounts for nurses' down-time and administrative tasks that need to be subtracted from the time spent on clinical tasks. Additionally, the productivity index may be used to schedule accurately the nurses' rota based on realistic hospital needs and use historic data to predict the upcoming demand over different ranges of time-horizons.

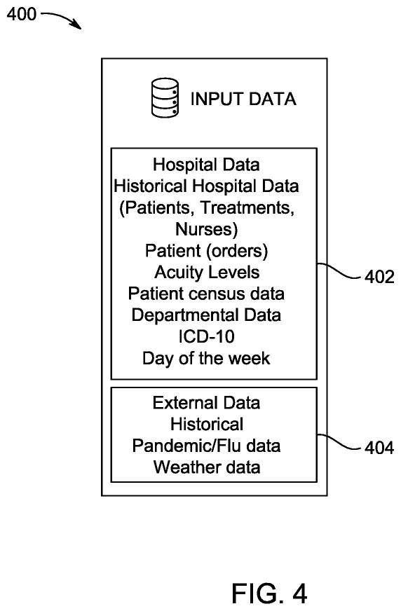

FIG. 4 illustrates a block diagram that shows various input data used in forecasting demand, in accordance with an example embodiment.

The input data 400 further comprises of hospital data 402 and external data 404. The hospital data further comprises of hospital historical data, work drivers, patient orders, acuity levels, patient census data, departmental data, ICD-10, and day of a week. In an example embodiment, the work drivers provides a forecast that caters to patient-dependent diversified needs.

In an example embodiment, in nursing services demand forecasting, hospitals rely on various types of data to accurately predict future demand for nursing services. These different types of hospital data provide valuable insights into patient demographics, healthcare trends, resource utilization, and other factors that impact nursing service demand. Mentioned below are the key types of hospital data required for nursing services demand forecasting.

Patient Admission Data: Patient admission data is fundamental for understanding the volume of patients entering the hospital. This data includes information such as the number of patients admitted within a specific period, the dates and times of admissions, and the departments or units to which they are admitted. By analyzing historical patient admission data, hospitals can identify trends, patterns, and variations in patient volumes, enabling them to anticipate future demand for nursing services accurately.

Length of Stay Data: Length of stay data provides insights into the duration of patients' hospital stays. Hospitals track the length of stay for each patient, which helps in estimating the average length of stay for different patient categories or medical conditions. This data is crucial for determining the nursing resources required to provide care for patients during their hospitalization. It helps in understanding the workload and staffing needs for various units or departments.

Patient Acuity Data: Patient acuity data refers to information about the complexity and intensity of care required by patients. It includes factors such as the severity of the medical condition, the level of intervention or treatment required, and the need for specialized nursing care. By collecting and analyzing patient acuity data, hospitals can assess the distribution of patient acuity levels and allocate nursing resources accordingly. This data helps in determining appropriate staffing ratios and nurse-to-patient ratios, ensuring that patients receive the necessary level of care.

Nursing Workload Data: Nursing workload data provides insights into the tasks and activities performed by nursing staff. This data includes information about the types of care provided, the time spent on direct patient care, administrative tasks, provider orders, and documentation requirements. Analyzing nursing workload data helps hospitals assess the workload distribution, identify potential bottlenecks or inefficiencies, and optimize staffing levels. It ensures that nurses can deliver high-quality care while maintaining manageable workloads.

Technology and Process Data: Technology and process data pertain to the implementation of healthcare technologies, workflows, and processes within the hospital. This data includes information about the adoption of electronic health records (EHRs), the use of digital tools, and any changes in care delivery models. Analyzing technology and process data helps hospitals understand how these factors impact nursing workflows, workload distribution, and resource utilization. It allows for adjustments in forecasting models to account for technological advancements and process changes.

Collaborative Data: Collaborative data involves the input and expertise of nursing managers, frontline nurses, and other healthcare professionals. These individuals provide valuable insights into unit-specific demands, upcoming projects, or changes in patient care requirements. Collaboration ensures that the forecasting process incorporates the collective knowledge and experience of the healthcare team, leading to more accurate demand forecasts.

Hence, nursing services demand forecasting in hospitals relies on a variety of data types. Patient admission data, length of stay data, patient acuity data, nursing workload data, external factors data, technology and process data, and collaborative data all play crucial roles in accurately predicting the future demand for nursing services. By analyzing and incorporating these different types of hospital data into forecasting models, hospitals can effectively allocate resources, plan staffing schedules, and ensure high-quality patient care.

In an another embodiment, the external data 404 are historical pandemic data and weather data.

In an example embodiment, the external data 404 plays a significant role in nursing services demand forecasting as it provides valuable insights into factors that influence the demand for nursing services beyond the hospital's internal operations. These external data sources help hospitals make informed projections and adjustments to their staffing and resource allocation. This essay will explore the key types of external data required for nursing services demand forecasting.

One important external data source is healthcare policies and regulations. Changes in healthcare policies, such as reimbursement models or regulations related to patient care, or facilities-based requirements can have a significant impact on nursing service demand. Hospitals need to monitor and analyze these policy changes to understand how they may influence patient admissions, care requirements, and resource allocation. By considering the evolving healthcare landscape, hospitals can make accurate projections about the demand for nursing services.

Population demographics are another crucial external data source. Factors such as population growth, aging populations, or shifts in local demographics can impact nursing service demand. As the population changes, the healthcare needs of different age groups or communities may fluctuate. By examining population demographics, hospitals can anticipate potential shifts in demand and adjust their nursing staffing levels and resource allocation accordingly.

Epidemiological data and disease outbreaks provide important external data for nursing services demand forecasting. The occurrence of infectious diseases or public health emergencies can significantly affect the demand for nursing services. Hospitals need to monitor and analyze data related to disease prevalence, outbreak patterns, and transmission rates. By considering the potential impact of epidemics or pandemics, hospitals can prepare for increased patient admissions, specialized care requirements, and heightened nursing service demand.

Economic indicators and trends also contribute to nursing services demand forecasting. Economic factors, such as unemployment rates, income levels, or healthcare spending, can influence healthcare utilization and patient admissions. By examining economic data, hospitals can gain insights into potential changes in patient volumes and adjust their nursing resources accordingly.

Technological advancements and innovation in healthcare are additional external data sources for nursing services demand forecasting. The adoption of new technologies, digital tools, or care delivery models can impact the demand for nursing services. Hospitals need to stay informed about technological advancements, such as the implementation of electronic health records or telehealth services. By considering the influence of these technological changes, hospitals can anticipate shifts in nursing workflows, resource utilization, and staffing needs.

Collaboration with other healthcare organizations and professionals is another valuable source of external data for nursing services demand forecasting. By sharing information and experiences, hospitals can gain insights into industry trends, best practices, and regional healthcare challenges. Collaborative data provides a broader perspective on nursing service demand, allowing hospitals to make more accurate projections based on shared knowledge and expertise.

Monitoring and analyzing local and national healthcare trends are essential for nursing services demand forecasting. Keeping track of healthcare utilization rates, patient satisfaction data, or healthcare outcome measures can provide valuable external insights. By considering these trends, hospitals can make informed decisions regarding nursing staffing levels, resource allocation, and service optimization.

Therefore, external data sources are vital for nursing services demand forecasting. Healthcare policies, population demographics, epidemiological data, economic indicators, technological advancements, collaboration with healthcare professionals, and monitoring healthcare trends all contribute to accurate projections of nursing service demand. By analyzing and incorporating these external factors into forecasting models, hospitals can effectively allocate resources, plan staffing schedules, and ensure high-quality patient care.

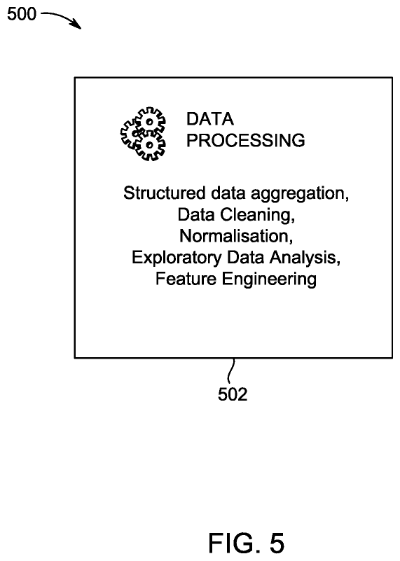

FIG. 5 illustrates a block diagram that shows different components in data processing module, in accordance with an example embodiment. In an embodiment, the data processing module 500 further comprises of components 502 such as structured data aggregation, data cleaning, normalization, exploratory data analysis and feature engineering.

In an example embodiment, in nursing services demand forecasting, measures of structured data aggregation, data cleaning, normalization, exploratory data analysis (EDA), and feature engineering are employed to ensure the accuracy and reliability of the forecasting models. Each step plays a crucial role in preparing and analyzing the data for effective forecasting. The measures are used in the context of nursing services demand forecasting in a hospital.

Structured data aggregation involves gathering relevant data from various sources within the hospital. This includes patient admission records, length of stay data, patient acuity levels, nursing workload data, and other pertinent information. By aggregating structured data, hospitals create a comprehensive dataset that serves as the foundation for forecasting models.

Data cleaning is a crucial step that involves identifying and rectifying errors, inconsistencies, and missing or redundant values within the collected data. This process ensures the data is accurate, complete, and reliable. In nursing services demand forecasting, data cleaning helps to address any discrepancies in the recorded data, such as incorrect patient admission dates or missing patient acuity information. By cleaning the data, hospitals can reduce biases and improve the quality of the dataset.

Normalization is performed to standardize the data and bring it to a common scale. This is particularly important when working with data that has different units of measurement or varying ranges. In nursing services demand forecasting, normalization is used to bring variables such as patient admissions, length of stay, and nursing workload to a standardized scale, enabling fair comparisons and accurate modeling.

Exploratory data analysis (EDA) is an essential step in understanding the characteristics and relationships within the data. It involves visualizing and summarizing the data to identify patterns, trends, and potential outliers. EDA helps hospitals gain insights into the distribution of variables, identify any correlations or dependencies between different data attributes, and understand the overall structure of the dataset. In nursing services demand forecasting, EDA can reveal relationships between patient admissions, length of stay, and nursing workload, providing valuable insights for modeling and forecasting.

Feature engineering is the process of transforming the existing data into meaningful features that enhance the forecasting models' predictive power. It involves creating new variables or combining existing ones to capture additional information or relationships within the data. In nursing services demand forecasting, feature engineering may involve creating variables that represent patient acuity levels, nursing-to-patient ratios, or the intensity of nursing care required. These engineered features can provide a more comprehensive representation of the nursing services demand and improve the accuracy of the forecasting models.

By employing structured data aggregation, data cleaning, normalization, exploratory data analysis, and feature engineering, hospitals can ensure the data used for nursing services demand forecasting is accurate, consistent, and relevant. These measures help to address data quality issues, standardize variables, uncover insights, and create meaningful features that contribute to more robust and accurate forecasting models. Ultimately, this aids hospitals in making informed decisions regarding resource allocation, staffing schedules, and delivering high-quality nursing care to meet the demands of patients effectively.

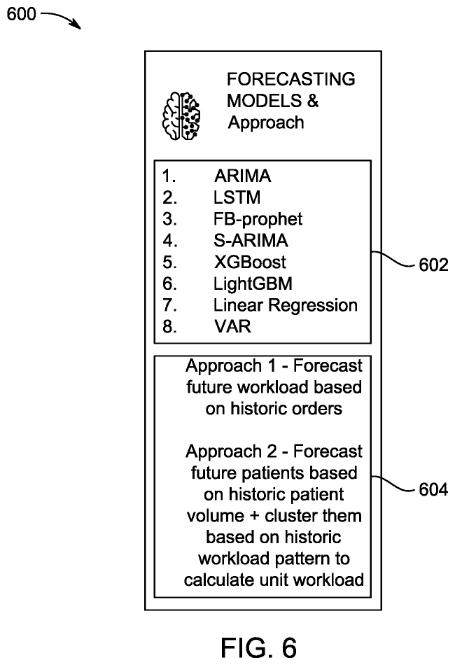

FIG. 6 illustrates a block diagram that shows various forecasting models, in accordance with an example embodiment. The forecasting models and approach 600 has the forecasting models 602 and approaches 604.

In an embodiment, the approaches 604 is forecasting future workload based on historic orders. Alternatively, forecasting future patients based on historic patient volume and clustering them based on historic workload pattern to calculate unit workload.

In an example embodiment, the various forecasting models 602 are ARIMA, LSTM, FB-prophet, S-ARIMA, XGBoost, LightGBM, Linear Regression and VAR.

In an example embodiment, in nursing services demand forecasting, various forecasting models can be employed to predict future demand accurately. Each model has its strengths and suitability depending on the specific characteristics of the data at the individual nursing unit level and the associated forecasting requirements. Below mentioned are how some forecasting models, namely ARIMA, LSTM, FB-prophet, S-ARIMA, XGBoost, LightGBM, Linear Regression, and VAR, may be utilized for nursing services demand forecasting in a hospital.

ARIMA (AutoRegressive Integrated Moving Average): ARIMA is a time series forecasting model that considers the autoregressive (AR), integrated (I), and moving average (MA) components of the data. ARIMA models are effective when the data exhibits a trend or seasonality. In nursing services demand forecasting, ARIMA can be used to capture the historical patterns and predict future demand based on past patient admission, length of stay, or nursing workload data. It can provide insights into short-term or long-term trends in nursing service demand.

LSTM (Long Short-Term Memory): LSTM is a type of recurrent neural network (RNN) that can effectively capture sequential dependencies and long-term patterns in time series data. In nursing services demand forecasting, LSTM models can be used to analyze the temporal relationships between variables such as patient admissions, length of stay, and nursing workload. LSTM can learn from historical data and make predictions based on the sequential nature of nursing service demand, accommodating complex patterns and dynamics.

FB-prophet: FB-prophet is a time series forecasting model. It is designed to handle time series data with various components, including trend, seasonality, and holidays. FB-prophet can be useful in nursing services demand forecasting as it can capture recurrent patterns, such as weekly or monthly fluctuations inpatient admissions or nursing workload. It also allows for the inclusion of external factors, such as public holidays or special events, which may impact nursing service demand.

S-ARIMA (Seasonal ARIMA): S-ARIMA is an extension of the ARIMA model that specifically accounts for seasonality in the data. It is suitable when the nursing service demand exhibits repetitive seasonal patterns, such as higher patient admissions during certain months or days of the week. By incorporating seasonality into the modeling process, S-ARIMA can provide accurate predictions for nursing service demand during specific periods or seasons.

XGBoost and LightGBM: XGBoost and LightGBM are powerful machine learning algorithms that belong to the gradient boosting framework. These models are highly versatile and can handle complex relationships and interactions in the data. In nursing services demand forecasting, XGBoost and LightGBM can be used to capture non-linear relationships between variables and make accurate predictions based on features such as patient admissions, length of stay, patient acuity, nursing workload, and external factors.

Linear Regression: Linear Regression is a simple yet effective statistical modeling technique that establishes a linear relationship between variables. In nursing services demand forecasting, Linear Regression can be used to predict nursing service demand based on historical data, such as patient admissions or nursing workload. It provides insights into the linear trend and relationship between input variables and nursing service demand, enabling hospitals to make informed decisions regarding resource allocation and staffing.

VAR (Vector Autoregression): VAR is a multivariate time series forecasting model that considers the relationship between multiple variables simultaneously. In nursing services demand forecasting, VAR models can capture the interdependencies between variables such as patient admissions, length of stay, patient acuity, and nursing workload. VAR models can provide insights into how changes in one variable affect the others, allowing hospitals to forecast nursing service demand based on a comprehensive understanding of the system dynamics.

These forecasting models offer a range of techniques to analyze and predict nursing services demand in a hospital setting. The selection of a particular model depends on factors such as the nature of the data, the presence of seasonality or trends, the need to incorporate external factors, and the complexity of the relationships between variables. By leveraging these models, hospitals can make accurate and informed decisions regarding resource allocation, staffing, and ensuring high-quality nursing care based on the forecasted demand.

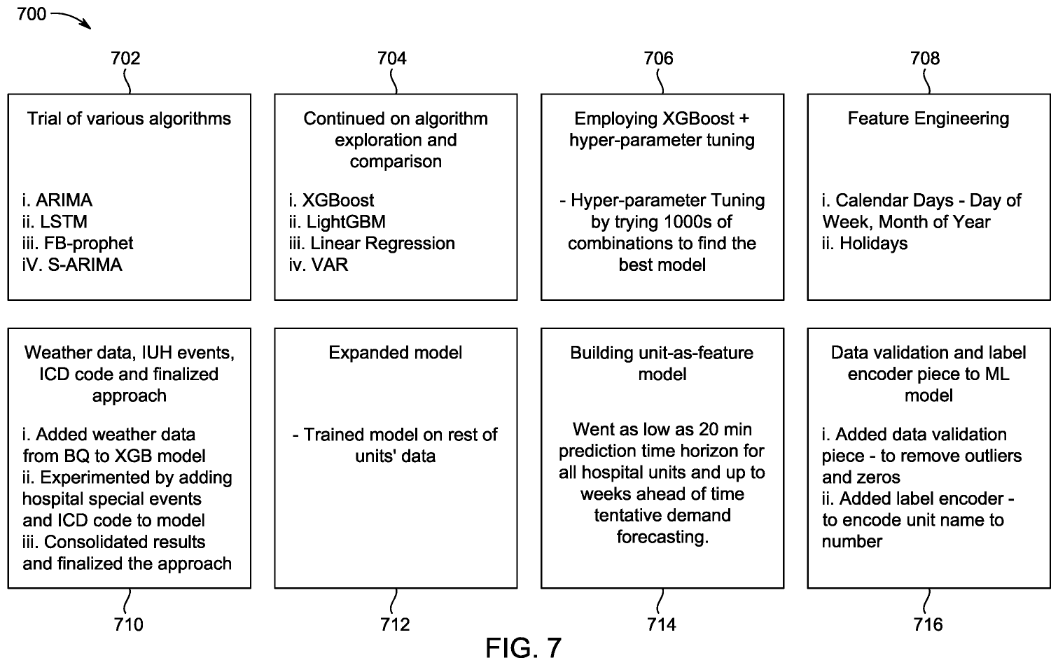

FIG. 7 illustrates a block diagram that shows various components in approaches to demand forecasting, in accordance with an example embodiment.

In an embodiment, for the approaches to demand forecasting 700 various phases are involved. The phases are trial of various algorithms 702 and further exploration and comparison 704. The algorithms tried may be ARIMA, LSTM, FB-prophet, XGBoost, LightGBM, Linear Regression and VAR.

In the next phase is employing XGBoost and in addition hyper-parameter tuning 706. Further, in an embodiment 1000s of combinations are tried to zero in on the best model using hyper parameter tuning.

In an example embodiment, feature engineering 708 is performed by considering calendar days, day of week, month of year and holidays.

Furthermore, weather data, hospital events and ICD (international classification of diseases) code are added and approach is finalized 710. In the next phase model is expanded 712 and trained on rest of units' data.

In an example embodiment, unit as a feature model is built 714. Here, the models are reduced at hospital levels and time intervals are limited to 4 and 24 hours.

In the final phase, data validation and label encoding are added to the model 716. The data validation is added to remove zeros and outliers and a label encoder is added encode unit name.

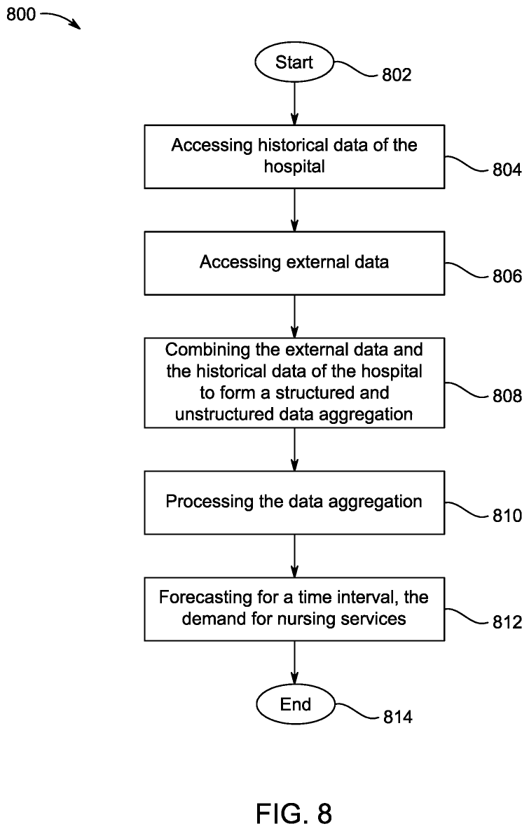

FIG. 8 illustrates a method 800 for forecasting the demand for nursing service, in accordance with an example embodiment. It will be understood that each block of the flow diagram of the method 800 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 204 of the evaluation system 200, employing an embodiment of the present disclosure and executed by a processor 202. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

The method 800 illustrated by the flow diagram of FIG. 800 for forecasting the demand for nursing services may start at step 802. The method 800 may include, at step 804, accessing historical data of the hospital. In an embodiment the historical data of a hospital may include but not limited to historical hospital data (patients, treatments, nurses), patient (orders), acuity levels, patient census data, departmental data, icd-10 and day of the week.

In an example embodiment, nursing services demand forecasting in a hospital setting relies on various types of historical hospital data to accurately predict future demand for nursing services. These different types of data provide valuable insights into patient demographics, healthcare utilization patterns, resource allocation, and other factors that impact nursing service demand. The key types of historical hospital data required for nursing services demand forecasting are mentioned below.

Patient admission data is one of the fundamental types of historical data required for nursing services demand forecasting. This data includes information such as the number of patients admitted to the hospital within a specific time period, the dates and times of admissions, and the departments or units to which they were admitted. By analyzing historical patient admission data, hospitals can identify trends, seasonal variations, and patterns in patient volumes, which are crucial for forecasting nursing service demand accurately.

Length of stay data provides insights into the duration of patients' hospital stays. Hospitals track the length of stay for each patient, which helps in estimating the average length of stay for different patient categories or medical conditions. This data is essential for understanding the patient flow and predicting the demand for nursing services over time. Hospitals can analyze historical length of stay data to identify trends, seasonal fluctuations, or changes in patient acuity that impact nursing service demand.

Patient acuity data refers to information about the complexity and intensity of care required by patients. It includes factors such as the severity of the medical condition, the level of intervention or treatment required, and the need for specialized nursing care. Historical patient acuity data provides valuable insights into the distribution of patient acuity levels over time. By analyzing this data, hospitals can identify trends, changes in patient acuity, and forecast the demand for nursing services accordingly.

Nursing workload data captures information about the tasks and activities performed by nursing staff triggered by provider orders. This drives the number of nursing hours needed to support the demand, may be used to estimate the number of patients cared for based on nurse hours available, and the types of care provided based on the intensity of the demand estimated. Historical nursing workload data allows hospitals to understand the workload distribution over time and identify patterns or fluctuations in nursing service demand. By analyzing historical workload data, hospitals can make informed decisions regarding staffing levels, nurse-to-patient ratios, and resource allocation for different units or departments.

Resource utilization data encompasses the usage of various resources within the hospital, including beds, equipment, and supplies including medications and therapies. Historical resource utilization data provides insights into the demand for hospital resources and its correlation with nursing service demand. By examining historical resource utilization patterns, hospitals can identify resource-intensive periods, predict future resource needs, and align nursing services accordingly.

Historical staffing data captures information about the number of nursing staff available during specific time periods. This data includes the number of registered nurses, licensed practical nurses, and other nursing professionals on duty as well as the support nursing staff to handle non-clinical tasks. By analyzing historical staffing data in conjunction with patient admission and workload data, hospitals can assess the impact of staffing levels on nursing service demand. This information helps in adjusting staffing schedules, optimizing workforce distribution, and ensuring appropriate nurse-to-patient ratios.

Historical outcomes and quality data provide insights into the effectiveness of nursing services and the quality of patient care provided. This data includes metrics such as patient satisfaction scores, healthcare-associated infection rates, readmission rates, and other quality outcome measures. By analyzing historical outcomes and quality data, hospitals can assess the relationship between nursing service delivery and patient outcomes. It helps in understanding the impact of nursing services on patient care and can guide forecasting models to consider quality indicators while predicting nursing service demand.

In conclusion, nursing services demand forecasting in a hospital setting relies on a range of historical hospital data. Patient admission data, length of stay data, patient acuity data, nursing workload data, resource utilization data, staffing data, and outcomes/quality data are crucial for accurate forecasting. By analyzing these different types of historical data, hospitals can identify patterns, trends, and factors that influence nursing service demand. This enables effective resource allocation, staffing planning, and ensures the provision of high-quality nursing care to meet the needs of patients effectively.

The method 800, at step 806, may include accessing external data. In an embodiment the external data may be historical pandemic data and weather data.

In some example embodiments, nursing services demand forecasting in a hospital setting requires external data sources to provide a broader perspective on factors influencing the demand for nursing services. External data complements internal hospital data and offers insights into population dynamics, healthcare policies, economic indicators, technological advancements, and collaborative information from other healthcare organizations. The key types of external data required for nursing services demand forecasting are mentioned below.

Population demographics play a crucial role in nursing services demand forecasting. Understanding population growth, age distribution, and changes in local demographics helps hospitals anticipate the healthcare needs of different age groups and communities. By analyzing population data, hospitals can project potential shifts in demand for nursing services and adjust their staffing and resource allocation accordingly.

Healthcare policies and regulations significantly impact nursing service demand. Changes in policies related to reimbursement models, healthcare coverage, or patient care guidelines can influence patient admissions, care requirements, and resource utilization. Hospitals need to monitor and analyze healthcare policies to anticipate how they may affect nursing service demand and make informed forecasts.

Economic indicators and trends are vital external data sources for nursing services demand forecasting. Factors such as unemployment rates, income levels, healthcare spending, and consumer behavior can affect healthcare utilization and patient admissions. Hospitals need to consider economic data to gain insights into potential changes in patient volumes and adjust their nursing resources accordingly.

Technological advancements and innovation in healthcare have a significant impact on nursing services demand. The adoption of new technologies, digital tools, telehealth services, or electronic health records can alter care delivery models and influence nursing service requirements. Hospitals should stay informed about technological advancements to understand their implications on nursing workflows, resource utilization, and staffing needs.

Collaboration with other healthcare organizations and professionals provides valuable external data for nursing services demand forecasting. Sharing information and experiences with peers in the industry allows hospitals to gain insights into best practices, regional healthcare challenges, and industry trends. Collaborative data offers a broader perspective on nursing service demand, enabling hospitals to make more accurate forecasts based on shared knowledge and expertise.

Monitoring and analyzing local and national healthcare trends contribute to nursing services demand forecasting. Keeping track of healthcare utilization rates, patient satisfaction data, healthcare outcome measures, and other relevant trends helps hospitals understand patterns and changes in nursing service demand. By considering these trends, hospitals can make informed decisions regarding staffing, resource allocation, and service optimization.

In conclusion, nursing services demand forecasting in a hospital setting requires external data sources to complement internal data. Population demographics, healthcare policies, economic indicators, technological advancements, collaboration with other healthcare organizations, and monitoring healthcare trends all provide valuable insights for accurate forecasting. By incorporating these external factors into forecasting models, hospitals can effectively allocate resources, plan staffing schedules, and ensure the delivery of high-quality nursing care to meet the demands of patients effectively.

The method 800, at step 808, may include combining the external data and the historical data of the hospital to form a structured data aggregation. In an embodiment, the external and historical data of the hospital is combined at step 808.

In an example embodiment, combining external data with the historical data of the hospital to form a structured data aggregation for forecasting nursing services demand can be achieved through several steps. The goal is to integrate relevant external data sources with internal hospital data to create a comprehensive and informative dataset. Mentioned below may an overview of the process.

Identify Relevant External Data Sources: First, identify the external data sources that are relevant to nursing services demand forecasting. These may include population demographic data, healthcare policy information, economic indicators, technological advancements in healthcare, collaborative data from other healthcare organizations, and local/national healthcare trends. Determine which data sources align with the forecasting objectives and are expected to have an impact on nursing service demand.

Obtain and Collect External Data: Once the relevant external data sources are identified, obtain access to the data. This may involve partnering with external organizations, utilizing public datasets, subscribing to data services, or collaborating with other healthcare institutions. Obtain the necessary permissions and agreements to collect and utilize the external data.

Preprocess and Cleanse External Data: Ensure that the collected external data is in a format compatible with the internal hospital data. Preprocess and clean the external data to address any inconsistencies, missing values, or errors. Standardize the external data to align with the internal data structure to facilitate seamless integration during the aggregation process.

Define Common Data Elements: To combine the external data with historical hospital data, define common data elements that serve as the basis for integration. Identify variables or attributes that can be shared between the external data and internal hospital data. For example, patient demographics, geographic regions, time periods, or unique identifiers can serve as common data elements for merging the datasets.

Merge the Datasets: Merge the cleaned external data with the historical hospital data based on the common data elements. Ensure that the merging process maintains data integrity and coherence. This can be achieved by using database management systems, statistical software, or programming languages that support data integration and aggregation. The result is a unified dataset that combines both internal and external data sources.

Perform Data Transformation and Normalization: Perform necessary data transformations and normalizations to bring the aggregated dataset to a common scale and format. This may involve converting data types, standardizing units of measurement, normalizing values, or applying statistical techniques to ensure compatibility and consistency across the variables.

Validate and Quality Check the Aggregated Data: Validate the aggregated dataset to ensure accuracy and reliability. Conduct quality checks to identify any inconsistencies, outliers, or data integrity issues. Validate the relationships between variables and assess the overall quality of the aggregated data to ensure its suitability for nursing services demand forecasting.

Store and Maintain the Aggregated Dataset: Store the aggregated dataset in a secure and accessible data repository. Ensure appropriate data governance practices are followed to maintain data privacy, security, and compliance. Regularly update and maintain the aggregated dataset to incorporate new historical data from the hospital and relevant external data sources.

By following these steps, combining external data with historical hospital data can result in a structured data aggregation for nursing services demand forecasting. This comprehensive dataset provides a holistic view of the factors influencing nursing service demand, allowing for more accurate and informed forecasting to support effective resource allocation, staffing decisions, and the delivery of high-quality nursing care.

The method 800, at step 810, may include processing the structured data aggregation.

The method 800, at step 812, may include forecasting for a time interval, the demand for nursing services.

In an example embodiment, forecasting the demand for nursing services in a hospital can be achieved through a systematic approach that incorporates historical data analysis, statistical modeling, and ongoing evaluation. The following steps outline the general process of forecasting nursing service demand.

Data Collection: Gather relevant historical data, including patient admission records, length of stay, acuity levels, nursing workload data, staffing information, and any other pertinent data that can impact nursing service demand. Ensure data accuracy and quality by conducting necessary data cleansing and validation processes.

Data Analysis: Perform exploratory data analysis to understand patterns, trends, and seasonality in the historical data. Identify key factors that influence nursing service demand, such as patient demographics, seasonal variations, and any external factors that impact healthcare utilization.

Statistical Modeling: Select an appropriate forecasting model based on the nature of the data and the forecasting objective. Common models used for nursing service demand forecasting include ARIMA, LSTM, FB-prophet, S-ARIMA, XGBoost, LightGBM, Linear Regression, and VAR. Apply the chosen model to the historical data to generate forecasts for nursing service demand.

Model Evaluation: Evaluate the performance of the forecasting model by comparing the forecasted values with the actual demand. Utilize appropriate evaluation metrics, such as mean absolute error (MAE), root mean squared error (RMSE), or mean absolute percentage error (MAPE), to assess the accuracy and reliability of the forecasts.

Refinement and Iteration: Refine the forecasting model based on the evaluation results and incorporate any feedback or adjustments. Iteratively improve the model by analyzing the forecast errors and making necessary modifications to enhance the accuracy of future predictions.

Consider External Factors: Integrate external factors, such as population demographics, healthcare policies, economic indicators, technological advancements, and collaborative data from other healthcare organizations, into the forecasting process. These external factors can provide additional insights and improve the accuracy of nursing service demand forecasts.

Continuous Monitoring and Updating: Nursing service demand forecasting is an ongoing process. Regularly monitor the accuracy of the forecasts and update the models as new data becomes available. Adjust the forecasting model parameters or incorporate additional data sources as needed to ensure the forecasts remain relevant and accurate.

Collaboration and Communication: Foster collaboration and communication among nursing administrators, healthcare professionals, and stakeholders to validate the forecasted demand and align staffing and resource allocation decisions based on the forecasts. Regularly review and discuss the forecast results to inform strategic planning and optimize nursing service delivery.

By following these steps and utilizing appropriate forecasting models and techniques, hospitals can achieve accurate and reliable predictions of nursing service demand. This enables proactive resource planning, efficient staffing allocation, and optimal delivery of high-quality nursing care to meet the needs of patients effectively.

In some example embodiments, a computer programmable product may be provided. The computer programmable product may comprise at least one non-transitory computer-readable storage medium having stored thereon computer-executable program code instructions that when executed by a computer, cause the computer to execute the method 800.

In an example embodiment, an apparatus for performing the method 800 of FIG. 8 above may comprise a processor (e.g., the processor 202) configured to perform some or each of the operations of the method 1600. The processor may, for example, be configured to perform the operations (802-812) by performing hardware implemented logical functions, executing stored instructions, or executing algorithms for performing each of the operations. Alternatively, the apparatus may comprise means for performing each of the operations described above. In this regard, according to an example embodiment, examples of means for performing operations (802-812) may comprise, for example, the processor 202 which may be implemented in the system 200 and/or a device or circuit for executing instructions or executing an algorithm for processing information as described above.

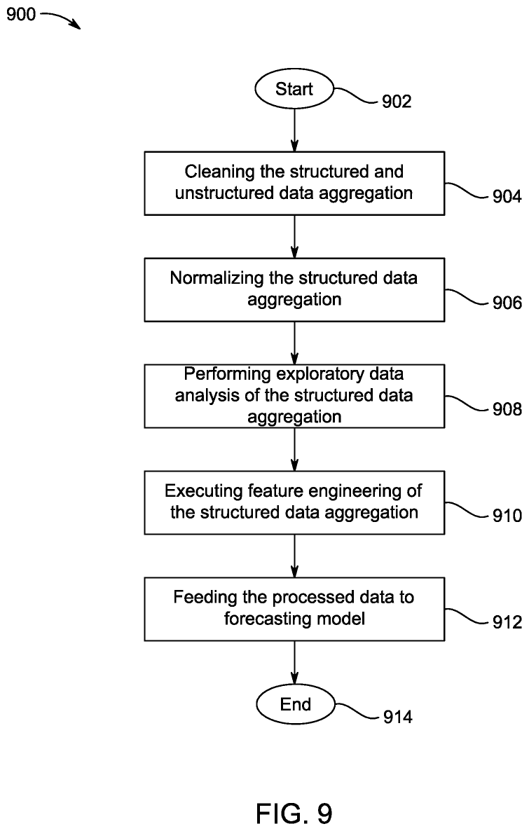

FIG. 9 illustrates a method 900 for demand input data processing, in accordance with an example embodiment. It will be understood that each block of the flow diagram of the method 900 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 204 of the evaluation system 200, employing an embodiment of the present disclosure and executed by a processor 202. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

The method 900 illustrated by the flow diagram of FIG. 900 for cleaning the structured data aggregation starts at step 902. In an embodiment, structured data aggregation involves combining relevant internal and external data sources to create a comprehensive dataset for analysis. In the context of nursing services demand forecasting, this step involves integrating historical hospital data, such as patient admission records, length of stay, acuity levels, and nursing workload data, with external data sources like population demographics, healthcare policies, and economic indicators. Aggregating data from various sources provides a holistic view of the factors influencing nursing service demand, enabling accurate and comprehensive forecasting.

The method at step 904, may include cleaning the structured data aggregation. In an embodiment, data cleaning is a critical step to ensure the accuracy and reliability of the dataset used for forecasting. It involves identifying and handling missing values, removing duplicates, correcting inconsistencies, and addressing outliers or errors in the data. In the context of nursing services demand forecasting, data cleaning ensures that the historical hospital data and external data sources are free from data quality issues that could potentially skew the forecasting results.

The method 900, at step 906, may include normalizing the structured data aggregation. In an example embodiment, normalization is the process of transforming numerical data to a common scale, typically between 0 and 1 or using z-scores, to eliminate differences in magnitude and ensure comparability between variables. In nursing services demand forecasting, normalization can be applied to standardize different metrics such as patient admission numbers, length of stay, and nursing workload. Normalization helps create a consistent framework for analyzing and interpreting the data, facilitating the identification of patterns and relationships between variables.

The method 900, at step 908, may include performing exploratory data analysis of the structured data aggregation. In an example embodiment, exploratory data analysis (EDA) involves examining the dataset visually and statistically to gain insights into its characteristics and uncover patterns, trends, and relationships. EDA techniques such as data visualization, summary statistics, and correlation analysis are applied to understand the distribution of variables, identify outliers or anomalies, detect seasonality or trends, and explore potential associations between nursing service demand and other factors. EDA helps in formulating hypotheses, validating assumptions, and guiding subsequent modeling decisions.

The method 900, at step 910, may include executing feature engineering of the structured data aggregation. In an example embodiment, feature engineering involves creating new variables or transforming existing variables to enhance the predictive power of the dataset. In the context of nursing services demand forecasting, feature engineering may involve creating lagged variables, such as previous month's nursing service demand, to capture temporal dependencies. It can also include creating interaction terms or deriving new variables that capture relevant relationships, such as the ratio of nursing staff to patient admissions. Feature engineering aims to extract meaningful information from the data and improve the forecasting models' ability to capture and predict nursing service demand accurately.

The method 900, at step 912, may include feeding the processed data to forecasting model. Further, structured data aggregation, data cleaning, normalization, exploratory data analysis, and feature engineering are essential steps in the process of forecasting demand for nursing services in a hospital. These steps ensure that the dataset used for forecasting is comprehensive, accurate, and appropriately prepared for analysis. The result is fed to the forecasting model.

In some example embodiments, a computer programmable product may be provided. The computer programmable product may comprise at least one non-transitory computer-readable storage medium having stored thereon computer-executable program code instructions that when executed by a computer, cause the computer to execute the method 900.

In an example embodiment, an apparatus for performing the method 900 of FIG. 9 above may comprise a processor (e.g., the processor 202) configured to perform some or each of the operations of the method 900. The processor may, for example, be configured to perform the operations (902-912) by performing hardware implemented logical functions, executing stored instructions, or executing algorithms for performing each of the operations. Alternatively, the apparatus may comprise means for performing each of the operations described above. In this regard, according to an example embodiment, examples of means for performing operations (902-912) may comprise, for example, the processor 202 which may be implemented in the system 200 and/or a device or circuit for executing instructions or executing an algorithm for processing information as described above.

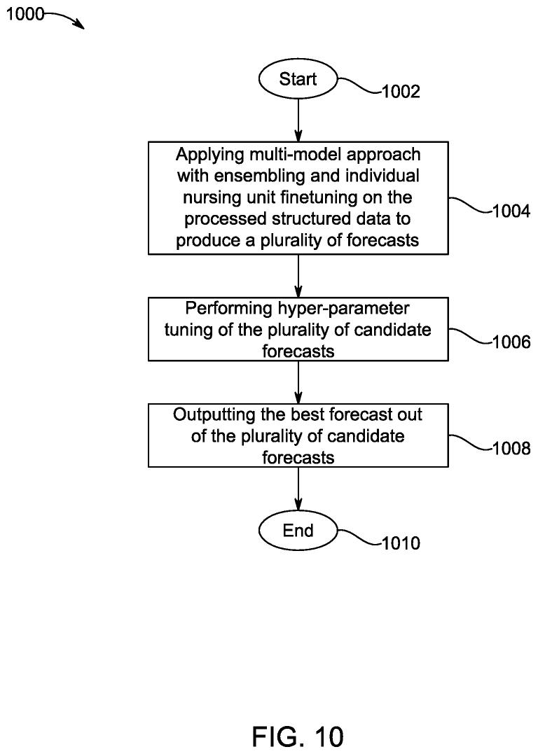

FIG. 10 illustrates a method 1000 for execution of Machine Learning model, in accordance with an example embodiment. It will be understood that each block of the flow diagram of the method 1000 may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory 204 of the evaluation system 200, employing an embodiment of the present disclosure and executed by a processor 202. As will be appreciated, any such computer program instructions may be loaded onto a computer or other programmable apparatus (for example, hardware) to produce a machine, such that the resulting computer or other programmable apparatus implements the functions specified in the flow diagram blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flow diagram blocks.

Accordingly, blocks of the flow diagram support combinations of means for performing the specified functions and combinations of operations for performing the specified functions for performing the specified functions. It will also be understood that one or more blocks of the flow diagram, and combinations of blocks in the flow diagram, may be implemented by special purpose hardware-based computer systems which perform the specified functions, or combinations of special purpose hardware and computer instructions.

The method 1000 illustrated by the flow diagram of FIG. 10 for execution of Machine Learning model may starts at step 1002.

The method 1000, at step 1004, may include applying XGBoost algorithm on the processed structured data to produce a plurality of forecasts. In an example embodiment, to use the XGBoost algorithm for forecasting nursing service demand, the historical hospital data, including relevant features such as patient admissions, length of stay, and nursing workload, is used as the training dataset. The algorithm learns from this data to capture the patterns and dependencies between the input features and the target variable, which is the demand for nursing services. The model is trained to predict future demand based on the historical patterns and relationships identified in the data.

The method 1000, at step 1006, may include performing hyper-parameter tuning of the plurality of candidate forecasts. In an example embodiment, hyperparameter tuning can be performed using various techniques, including grid search, random search, or more advanced optimization algorithms like Bayesian optimization. By systematically exploring different combinations of hyperparameter values and evaluating the performance of the model using appropriate evaluation metrics, such as mean absolute error (MAE) or root mean squared error (RMSE), the optimal set of hyperparameters can be identified.

The process of hyperparameter tuning aims to find the best trade-off between model complexity and generalization performance. It helps in fine-tuning the XGBoost model for forecasting nursing service demand, resulting in more accurate predictions and better capturing of the underlying patterns in the data.

The method 1000, at step 1008, may include outputting the best forecast out of the plurality of candidate forecasts

In some example embodiments, a computer programmable product may be provided. The computer programmable product may comprise at least one non-transitory computer-readable storage medium having stored thereon computer-executable program code instructions that when executed by a computer, cause the computer to execute the method 1000.

In an example embodiment, an apparatus for performing the method 1000 of FIG. 10 above may comprise a processor (e.g., the processor 202) configured to perform some or each of the operations of the method 1000. The processor may, for example, be configured to perform the operations (1002-1008) by performing hardware implemented logical functions, executing stored instructions, or executing algorithms for performing each of the operations. Alternatively, the apparatus may comprise means for performing each of the operations described above. In this regard, according to an example embodiment, examples of means for performing operations (1002-1010) may comprise, for example, the processor 202 which may be implemented in the system 200 and/or a device or circuit for executing instructions or executing an algorithm for processing information as described above.

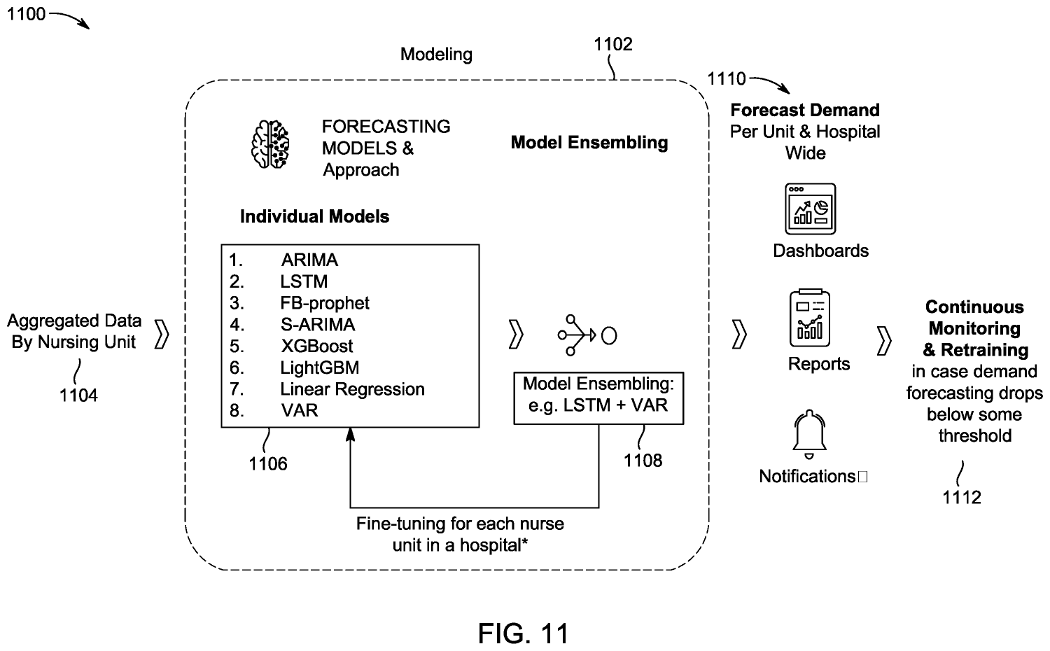

FIG. 11 shows a block diagram for process flow of nursing services demand prediction, in accordance with an example embodiment. Aggregated data by nursing unit 1104 is forwarded to the modelling 102 for forecasting.

Parallelization and independent model tuning are two techniques used to achieve faster processing in machine learning (ML) models. Parallelization involves dividing a task into smaller subtasks that can be executed simultaneously on multiple processing units. By splitting the workload across multiple units, parallelization significantly reduces computation time.

Model parallelism, on the other hand, divides the model itself into subparts, each processed independently by different units. This approach is beneficial for models with complex architectures, as it allows parallel computation of different parts of the model. By exploiting the power of hardware architectures, parallelization accelerates ML computations, particularly for computationally intensive tasks.

Independent model tuning is another technique used to enhance ML model performance and speed. ML models often have hyperparameters, which are configuration settings that influence the model's learning process and performance. Independent model tuning involves optimizing these hyperparameters independently for each unit or instance of a model.

Independent model tuning allows for exploring different hyperparameter configurations in parallel and leveraging the collective knowledge of multiple models. This approach can significantly enhance predictive accuracy or efficiency by effectively searching the hyperparameter space.

Combining parallelization techniques with independent model tuning can achieve speed improvements in ML models. By parallelizing tasks across multiple processing units and independently tuning models or model instances, both computational efficiency and overall performance can be enhanced.

The modeling 102 comprises of individual models 1106 and ensembling at least come of the individual models 1106. There are several models employed, the models are but not limited to ARIMA, S-ARIMA, LSTM, FB-prophet, XGBoost, LightGBM, and VAR. The aggregated date by nursing unit 1104 is processed by one or more individual models 1106 and is further the model ensembling 1108 is performed on the outputs of the individual models 1106.

Ensembling 1108 technique in machine learning involves combining multiple models to make more accurate predictions or decisions than any single model could achieve on its own. It leverages the diversity and collective wisdom of different models to create a stronger and more robust predictive system. In an example embodiment, ensembling may speed up the process from a plurality of hours down to minutes.

The concept of ensembling stems from the idea that individual models may have their strengths and weaknesses. By combining multiple models, the goal is to capitalize on their respective strengths and mitigate their weaknesses, resulting in a more reliable and accurate prediction. Ensembling 1108 may be applied to various machine learning tasks, such as classification, regression, and even unsupervised learning.

One common approach to ensembling is called “model averaging” or “model voting.” In this method, several base models are trained independently on the same dataset using different algorithms or variations in the training process. Each model generates its predictions, and these predictions are combined to produce the final ensemble prediction. The combination may be achieved through various techniques, including simple averaging, weighted averaging, or majority voting.

The key intuition behind model averaging is that by combining diverse models, the ensemble may capture a broader range of patterns and insights from the data. Different models may excel in different areas or have different perspectives on the underlying patterns, and their combination can lead to a more comprehensive understanding of the data. This diversity helps to reduce bias and increase the overall accuracy of predictions.

Another popular ensemble technique is called “bagging” (short for bootstrap aggregating). Bagging involves training multiple models on different subsets of the training data, where each subset is sampled with replacement. By creating diverse training sets, bagging promotes model variance and reduces the impact of individual data points or outliers. The final prediction is obtained by aggregating the predictions of all the models, usually through averaging.

In addition to bagging, there is another ensemble method called “boosting.” Boosting is an iterative process where models are trained sequentially, with each model attempting to correct the mistakes made by its predecessors. The models are typically trained on weighted versions of the training data, with more weight assigned to the instances that were misclassified by earlier models. The final prediction is a weighted combination of the predictions made by all the models in the ensemble.

Ensembling can also be extended to more sophisticated techniques, such as stacking or meta-learning. Stacking involves training multiple models on the same dataset and then training a meta-model on their predictions. The meta-model learns to combine the base models' outputs to make the final prediction. This approach allows the ensemble to capture both the individual models' insights and the higher-level patterns in their predictions.

Ensembling enhances predictive performance and generalization. By leveraging the collective knowledge of multiple models, ensembling can often achieve higher accuracy than any single model. It is particularly effective when the base models are diverse, meaning they have different underlying assumptions, architectures, or learning algorithms.

In summary, ensembling is a powerful technique in machine learning that combines the predictions of multiple models to improve accuracy and robustness. By harnessing the diversity and collective wisdom of different models, ensembling can provide more reliable predictions and overcome the limitations of individual models. It offers a flexible framework for improving the performance of machine learning systems and has become a valuable tool for various tasks and applications in the field.

Furthermore, a clustering technique is employed to find the best-fit of the ensemble machine learning models to each individual nursing unit. Thereby, customizing and tuning the predictive model for each nursing unit specific characteristics.

Additionally, the accuracy may be monitored using a range of regression metrics on the predicted versus actual demand, in an embodiment Mean Average Percentage Error (MAPE) is employed.

Further, after ensembling output which is the forecast demand which may be per unit and hospital wide is output 1110. Also, the forecast demand output 1110 may be in form of dashboards, reports, or alerts. A person skilled in the art may not be limited to the said output forms and may employ a plurality of the output forms. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

It is to be understood that the above description is intended to be illustrative, and not restrictive. For example, the above-discussed embodiments may be used in combination with each other. Many other embodiments will be apparent to those of skill in the art upon reviewing the above description.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions, and improvements to the embodiments described above are possible. It is contemplated that these variations, modifications, additions, and improvements fall within the scope of the invention.

What is claimed is:

1. A computer-implemented method for forecasting demand for nursing services in a hospital comprising:
accessing historical data of the hospital;
accessing external data;
combining the external data and the historical data of the hospital to form a structured data aggregation;
processing the structured data aggregation;
performing a plurality of forecasts for a time interval, the demand for nursing services based on the processed structured data aggregation; and
ensembling the plurality of forecasts.

2. The computer-implemented method of claim 1, wherein the historical data of the hospital comprises of at least patient-level work drivers, acuity levels, patients' census data, departmental data, ICD-10 data, and day of a week.

3. The computer-implemented method of claim 1, wherein the external data comprises of at least historical pandemic data, seasonal communicable disease data and weather data.

4. The computer-implemented method of claim 3, wherein the processing the structured data aggregation comprising:
cleaning the structured data aggregation;
normalizing the structured data aggregation;
performing exploratory data analysis of the structured data aggregation; and
executing feature engineering of the structured data aggregation.

5. The computer-implemented method of claim 1, wherein the time interval comprises 4 hours or 24 hours.

6. The method of claim 1, wherein the forecasting is performed by applying XGBoost algorithm on the processed structured data aggregation to produce a plurality of candidate forecasts.

7. The computer-implemented method of claim 1, further comprising obtaining an optimum forecast by hyper-parameter tuning of the plurality of candidate forecasts.

8. The computer-implemented method of claim 7, wherein the forecasting is online and real-time.

9. The computer-implemented method of claim 8, wherein the forecasting further comprises clustering based on historical data.

10. The computer-implemented method of claim 1, wherein the plurality of forecasts comprise VAR and LSTM.

11. A computer system for forecasting demand for nursing services in a hospital comprising, the computer system comprising: one or more computer processors, one or more computer readable memories, one or more computer readable storage devices, and program instructions stored on the one or more computer readable storage devices for execution by the one or more computer processors via the one or more computer readable memories, the program instructions comprising:
accessing historical data of the hospital;
accessing external data;
combining the external data and the historical data of the hospital to form a structured data aggregation;
processing the structured data aggregation; and
forecasting for a time interval, the demand for nursing services based on the processed structured data aggregation.

12. The system of claim 10, wherein the historical data of the hospital comprises of at least patient orders, acuity levels, patients' census data, departmental data, ICD-10 data, and day of a week.

13. The system of claim 10, wherein the external data comprises of at least historical pandemic data and weather data.

14. The system of claim 12, wherein the processing the structured data aggregation comprising:
cleaning the structured data aggregation;
normalizing the structured data aggregation;
performing exploratory data analysis of the structured data aggregation; and
executing feature engineering of the structured data aggregation.

15. The system of claim 10, wherein the time interval comprises 4 hours or 24 hours.

16. The system of claim 10, wherein the forecasting is performed by applying XGBoost algorithm on the processed structured data aggregation to produce a plurality of candidate forecasts.

17. The system of claim 10, further comprising obtaining an optimum forecast by hyper-parameter tuning of the plurality of candidate forecasts.

18. The system of claim 16, wherein the forecasting is online and real-time.

19. The system of claim 17, wherein the forecasting further comprises clustering based on historical data.

20. A non-transitory computer-readable storage medium having stored thereon computer executable instruction which when executed by one or more processors, cause the one or more processors to carry out operations for forecasting demand for nursing services in a hospital comprising, the operations comprising perform the operations comprising:
accessing historical data of the hospital;
accessing external data;
combining the external data and the historical data of the hospital to form a structured data aggregation;
processing the structured data aggregation; and
forecasting for a time interval, the demand for nursing services based on the processed structured data aggregation.
